(19) United States
(12) Patent Application Publication
(10) Pub. No.: US 2025/0140414 A1
(43) Pub. Date: May 1, 2025

(54) REPRESENTATIVE DATASETS FOR BIOMEDICAL MACHINE LEARNING MODELS

(71) Applicant: Ventana Medical Systems, Inc., Tucson, AZ (US)

(72) Inventors: Ipshita Bhattacharya, San Jose, CA (US); Christoph Guetter, Alameda, CA (US); Uday Kurkure, Sunnyvale, CA (US); Mohammad Saleh Miri, San Jose, CA (US)

(73) Assignee: Ventana Medical Systems, Inc., Tucson, AZ (US)

(21) Appl. No.: 19/009,479

(22) Filed: Jan. 3, 2025

Related U.S. Application Data
(63) Continuation of application No. PCT/US2023/028458, filed on Jul. 24, 2023.

(51) Int. Cl. G16H 50/20

(52) U.S. Cl. CPC G16H 50/20

(57) ABSTRACT

Embodiments disclosed herein generally relate to representative datasets for biomedical machine learning models. Particularly, aspects of the present disclosure are directed to identifying a representative distribution of characteristics for a disease, generating a dataset comprising a set of biomedical images, wherein the dataset has a distribution of the characteristics that corresponds to the representative distribution of the characteristics for the disease, processing the dataset using a trained machine learning model, and outputting a result of the processing, wherein the result corresponds to a prediction that a biomedical image of the dataset includes a depiction of a set of tumor cells or other structural and/or functional biological entities associated with the disease, the biomedical image is associated with a diagnosis of the disease, the biomedical image is associated with a classification of the disease, and/or the biomedical image is associated with a prognosis for the disease.

### CROSS-REFERENCE TO RELATED APPLICATIONS

The present application is a continuation of International Application No. PCT/US2023/028458, filed on Jul. 24, 2023, which claims priority to U.S. Provisional Patent Application No. 63/392,394 filed on Jul. 26, 2022, each of which are hereby incorporated by reference in their entireties for all purposes.

### BACKGROUND

Digital pathology involves scanning slides of samples (e.g., tissue samples, blood samples, urine samples, etc.) into digital images. The sample can be stained such that select proteins (antigens) in cells are differentially visually marked relative to the rest of the sample. The target protein in the specimen may be referred to as a biomarker. Digital images with one or more stains for biomarkers can be generated for a tissue sample. These digital images may be referred to as histopathological images. Histopathological images can allow visualization of the spatial relationship between tumorous and non-tumorous cells in a tissue sample. Image analysis may be performed to identify and quantify the biomarkers in the tissue sample. The image analysis can be performed by computing systems or pathologists to facilitate characterization of the biomarkers (e.g., in terms of presence, size, shape and/or location) so as to inform (for example) diagnosis of a disease, determination of a treatment plan, or assessment of a response to a therapy. In addition to histopathological images, other biomedical images such as magnetic resonance imaging (MRI) images, computed tomography (CT) images, and ultrasound images may be similarly analyzed by computing systems or technicians to facilitate characterization of areas of interest to inform (for example) diagnosis of a disease, determination of a treatment plan, or assessment of a response to a therapy.

However, there may be regulations regarding accuracy, bias, explainability, and efficacy of predictions made by machine-learning models trained to characterize the biomarkers or areas of interest that the machine-learning models must meet before being approved for clinical use. Creating machine-learning models that meet the regulations may be difficult, and machine-learning models that do not meet the regulations may lead to prediction inaccuracies.

### SUMMARY

In various embodiments, a computer-implemented method is provided that includes identifying a representative distribution of characteristics for a disease and generating a dataset that includes a set of biomedical images. A distribution of the characteristics for dataset can correspond to the representative distribution of the characteristics for the disease. The dataset is processed using a trained machine learning model and a result of the processing is output. The result can correspond to a prediction that a biomedical image of the dataset includes a depiction of a set of tumor cells or other structural and/or functional biological entities associated with the disease, the biomedical image is associated with a diagnosis of the disease, the biomedical image is associated with a classification of the disease, and/or the biomedical image is associated with a prognosis for the disease. The prediction can characterize a presence of, quantity of and/or size of the set of tumor cells or the other structural and/or functional biological entities, the diagnosis of the disease, the classification of the disease, and/or the prognosis of the disease.

In some embodiments, prior to processing the dataset using the trained machine learning model, the computer-implemented method can include determining the distribution of the characteristics in the dataset, determining a difference between the distribution and the representative distribution, and modifying the dataset based on the difference.

In certain embodiments, the computer-implemented method can involve identifying a threshold criterion for a metric associated with the prediction, determining that the metric satisfies the threshold criterion, and availing the trained machine learning model for subsequent processing of biomedical images. Additionally or alternatively, the computer-implemented method can involve determining that the metric does not satisfy the threshold criterion, availing the trained machine learning model for subsequent processing of biomedical images, performing the subsequent processing of another set of biomedical images using the trained machine learning model, and outputting another result of the subsequent processing. The other result can indicate the prediction and a confidence level of the prediction based on the metric not satisfying the threshold criterion.

In some embodiments, the characteristics can include clinical characteristics (e.g., variants of the disease, a relapse rate, one or more international prognostic index risk factors, and/or one or more demographic factors). The characteristics can also include technical characteristics (e.g., one or more types of data acquisition methods, one or more types of scanner, and/or one or more types of staining protocol).

In some embodiments, a computer-implemented method involves identifying a representative distribution of characteristics for a disease and generating a dataset that includes a set of biomedical images. A distribution of the characteristics for the dataset corresponds to the representative distribution of the characteristics for the disease. The machine-learning model is trained using the dataset. An outcome of the training corresponds to a trained machine learning model. The trained machine learning model is availed to process a biomedical image.

In some embodiments, prior to training the machine-learning model, the computer-implemented method can include determining that biomedical images including the set of biomedical images excludes representative data for a characteristic of the characteristics. A notification can be output indicating the plurality of biomedical images excludes the representative data. An adjustment can be received for a value of the characteristic for the representative distribution and the dataset can be generated with the adjustment for the value.

In certain embodiments, the dataset is a first dataset and the computer-implemented method can involve processing a second dataset of biomedical images using the trained machine learning model and outputting a result of the processing. The result can correspond to a prediction that a biomedical image of the second dataset includes a depiction of a set of tumor cells or other structural and/or functional biological entities associated with the disease, the biomedical image is associated with a diagnosis of the disease, the biomedical image is associated with a classification of the disease, and/or the biomedical image is associated with a prognosis for the disease.

In some embodiments, a computer-implemented method is provided that involves identifying a biomedical image of a slice of specimen. The biomedical image is associated with a disease. The biomedical image is input to a trained machine learning model. The trained machine learning model was trained using a dataset having a distribution of characteristics that corresponds to a representative distribution of characteristics for the disease. A result of the trained machine learning model is received. The result corresponds to a prediction that the biomedical image includes a depiction of a set of tumor cells or other structural and/or functional biological entities associated with the disease, the biomedical image is associated with a diagnosis of the disease, the biomedical image is associated with a classification of the disease, and/or the biomedical image is associated with a prognosis for the disease.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTIONS OF THE DRAWINGS

Aspects and features of the various embodiments will be more apparent by describing examples with reference to the accompanying drawings, in which:

FIG. 1 shows an exemplary computing system for generating a representative dataset for training and using a machine-learning model;

FIG. 2 illustrates an exemplary process of training and using a machine-learning model with a representative dataset;

FIG. 3 shows exemplary characteristics of a representative dataset for training or testing a machine-learning model;

FIG. 4 shows a graph of exemplary results of a gap analysis between a representative distribution and an actual distribution in a generated dataset for a characteristic of a disease; and

FIG. 5 shows a graph of exemplary performance metrics for a machine-learning model tested using a representative dataset.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

### I. Overview

The present disclosure describes techniques for processing biomedical images using machine-learning models. More specifically, some embodiments of the present disclosure provide processing biomedical images by machine-learning models trained or tested using representative datasets.

Digital pathology may involve the interpretation of digitized pathology images in order to correctly diagnose subjects and guide therapeutic decision making. In digital pathology solutions, image-analysis workflows can be established to automatically detect or classify biological objects of interest e.g., positive, negative tumor cells, etc. An exemplary digital pathology solution workflow includes obtaining tissue slides, scanning preselected areas or the entirety of the tissue slides with a digital image scanner (e.g., a whole slide image (WSI) scanner) to obtain digital images, performing image analysis on the digital image using one or more image analysis algorithms, and potentially detecting, quantifying (e.g., counting or identify object-specific or cumulative areas of) each object of interest based on the image analysis (e.g., quantitative or semi-quantitative scoring such as positive, negative, medium, weak, etc.).

During imaging and analysis, regions of a digital pathology image may be segmented into target regions (e.g., positive and negative tumor cells) and non-target regions (e.g., normal tissue or blank slide regions). Each target region can include a region of interest that may be characterized and/or quantified. Machine-learning models can be developed to segment and characterize the target regions. But, a machine-learning model may only be approved for clinical use, for example to assist diagnoses and treatment of diseases, if the machine-learning model can be proven to meet regulatory standards. The regulatory standards may provide thresholds for performance metrics, such as accuracy, bias, precision, and sensitivity, of the predictions of the machine-learning model.

In some embodiments, a representative distribution of characteristics of a disease is identified. The representative distribution indicates a distribution of clinical characteristics for the disease according to historical clinical data as well as technical characteristics for equipment used in generating digital pathology images of the disease that may result in better performance of a machine-learning model. A dataset of biomedical images is then generated that has a distribution of characteristics that corresponds to the representative distribution of the characteristics. The distribution may accurately correspond to the representative distribution in a manner such that one or more measured properties of the distribution of the dataset are sufficiently close to the representative distribution. For example, the distribution may accurately correspond to the representative distribution if the distribution is within a predefined absolute value from or within a predefined percentage from the representative distribution. In addition, the distribution may accurately correspond to the representative distribution if an integral (or normalized integral) of an overlap of the distribution and the representative distribution exceeds a threshold. If the dataset cannot be generated such that one or more measured properties of the distribution is sufficiently close to the representative distribution, an output may be generated that indicates the lack of representative data for one or more characteristics. A user can then provide an adjustment for values of the one or more characteristics that can be used rather than the representative distribution. Accordingly, deviations from the representative distribution can be known. The dataset may be used to train a machine-learning model to detect tumor cells or other structural and/or functional biological entities of the disease. Alternatively, the dataset may be used to test or validate a previously trained machine learning model. Once trained, tested, or validated in accordance with particular performance metrics or regulatory standards, the machine-learning model can be availed for subsequent processing of additional biomedical images.

Using a representative dataset during training may provide a trained machine learning model with more accurate detection of tumor cells other structural and/or functional biological entities of a disease. In addition, as mentioned, deviations from the representative distribution may be known during the training, so predictions for biomedical images having a deviated characteristic can be accompanied with an indication that the training may not have been comprehensive for that characteristic. Also, using the representative dataset during testing or validation can provide performance metrics for predictions output by the trained machine learning model. As a result, the trained machine learning model may not be made available for clinical use until the performance metrics satisfy threshold criterions. Once the trained machine learning model is made available for clinical use and since the trained machine learning model is known to meet the performance metrics, the predictions made by the trained machine learning model may be sufficiently accurate, which can provide improved diagnosis facilitation and treatment recommendation based on predictions. In addition, a confidence level may be output with the prediction if the trained machine learning model is determined to not meet a particular threshold for a performance metric. As a result, a user may be more informed about the performance of the trained machine learning model. Thus, the representative dataset may additionally provide improved diagnosis facilitation and treatment recommendation based on predictions made by the machine-learning model and the corresponding confidence levels.

### II. Computing Environment

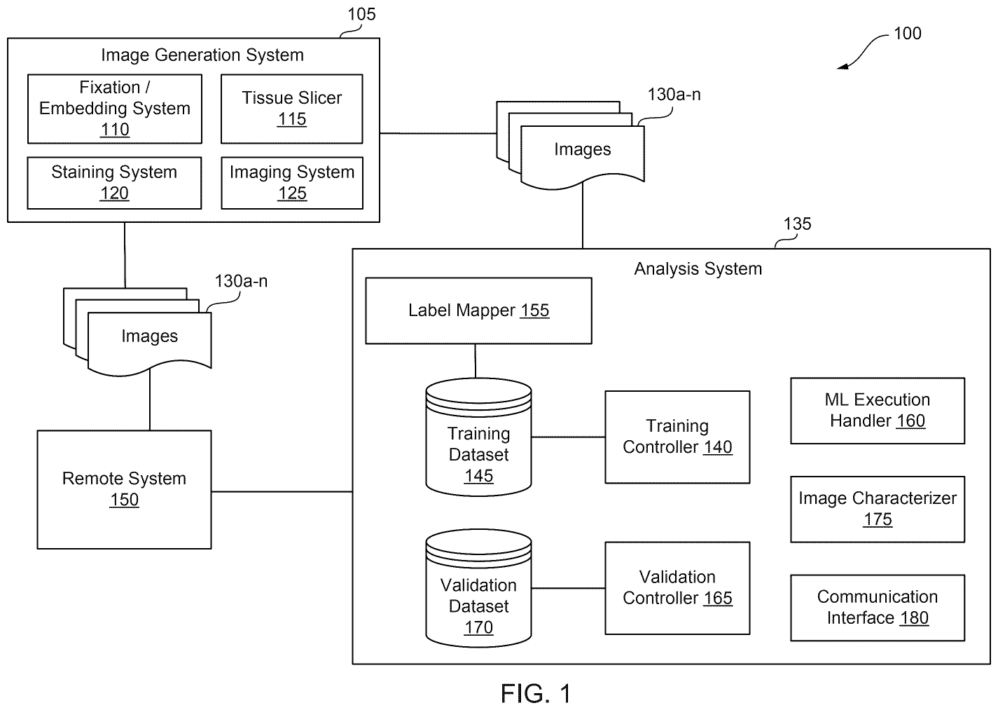

FIG. 1 shows an exemplary computing system 100 for generating a representative dataset for training and using a machine-learning model. Images are generated at an image generation system 105. The images may be biomedical images, such as histopathological images, computed tomography (CT) images, magnetic resonance imaging (MRI) images, ultrasound images, or any other suitable biomedical images. For histopathological images, a fixation/embedding system 110 fixes and/or embeds a tissue sample (e.g., a sample including at least part of at least one tumor) using a fixation agent (e.g., a liquid fixing agent, such as a formaldehyde solutiFneon) and/or an embedding substance (e.g., a histological wax, such as a paraffin wax and/or one or more resins, such as styrene or polyethylene). Each slice may be fixed by exposing the slice to a fixating agent for a predefined period of time (e.g., at least 3 hours) and by then dehydrating the slice (e.g., via exposure to an ethanol solution and/or a clearing intermediate agent). The embedding substance can infiltrate the slice when it is in liquid state (e.g., when heated).

A tissue slicer 115 then slices the fixed and/or embedded tissue sample (e.g., a sample of a tumor) to obtain a series of sections, with each section having a thickness of, for example, 4-5 microns. Such sectioning can be performed by first chilling the sample and the slicing the sample in a warm water bath. The tissue can be sliced using (for example) using a vibratome or compresstome.

Because the tissue sections and the cells within them are virtually transparent, preparation of the slides typically includes staining (e.g., automatically staining) the tissue sections in order to render relevant structures more visible. In some instances, the staining is performed manually. In some instances, the staining is performed semi-automatically or automatically using a staining system 120.

The staining can include exposing an individual section of the tissue to one or more different stains (e.g., consecutively or concurrently) to express different characteristics of the tissue. For example, each section may be exposed to a predefined volume of a staining agent for a predefined period of time. A duplex assay includes an approach where a slide is stained with two biomarker stains. A singleplex assay includes an approach where a slide is stained with a single biomarker stain. A multiplex assay includes an approach where a slide is stained with two or more biomarker stains.

One exemplary type of tissue staining is histochemical staining, which uses one or more chemical dyes (e.g., acidic dyes, basic dyes) to stain tissue structures. Histochemical staining may be used to indicate general aspects of tissue morphology and/or cell microanatomy (e.g., to distinguish cell nuclei from cytoplasm, to indicate lipid droplets, etc.). One example of a histochemical stain is hematoxylin and eosin (H&E). Other examples of histochemical stains include trichrome stains (e.g., Masson's Trichrome), Periodic Acid-Schiff (PAS), silver stains, and iron stains. The molecular weight of a histochemical staining reagent (e.g., dye) is typically about 500 kilodaltons (kD) or less, although some histochemical staining reagents (e.g., Alcian Blue, phosphomolybdic acid (PMA)) may have molecular weights of up to two or three thousand kD. One case of a high-molecular-weight histochemical staining reagent is alpha-amylase (about 55 kD), which may be used to indicate glycogen.

Another type of tissue staining is immunohistochemistry (IHC, also called “immunostaining”), which uses a primary antibody that binds specifically to the target antigen of interest (also called a biomarker). IHC may be direct or indirect. In direct IHC, the primary antibody is directly conjugated to a label (e.g., a chromophore or fluorophore). In indirect IHC, the primary antibody is first bound to the target antigen, and then a secondary antibody that is conjugated with a label (e.g., a chromophore or fluorophore) is bound to the primary antibody. The molecular weights of IHC reagents are much higher than those of histochemical staining reagents, as the antibodies have molecular weights of about 150 kD or more.

The sections may be then be individually mounted on corresponding slides, which an imaging system 125 can then scan or image to generate raw digital-pathology, or histopathological, images. The histopathological images may be included in images 130a-n, which are biomedical images. Each section may be mounted on a slide, which is then scanned to create a digital image that may be subsequently examined by digital pathology image analysis and/or interpreted by a human pathologist (e.g., using image viewer software). The pathologist may review and manually annotate the digital image of the slides (e.g., tumor area, necrosis, etc.) to enable the use of image analysis algorithms to extract meaningful quantitative measures (e.g., to detect and classify biological objects of interest). Conventionally, the pathologist may manually annotate each successive image of multiple tissue sections from a tissue sample to identify the same aspects on each successive tissue section.

The computing system 100 can include an analysis system 135 to train and execute a machine-learning model. Examples of the machine-learning model can be a deep convolutional neural network, a U-Net, a V-Net, a residual neural network, or a recurrent neural network. The machine-learning model may be trained and/or used to (for example) predict whether a biomedical image includes a depiction of a set of tumor cells or other structural and/or functional biological entities associated with a disease, whether the biomedical image is associated with a diagnosis of the disease, whether the biomedical image is associated with a classification (e.g., stage, subtype, etc.) of the disease, and/or the biomedical image is associated with a prognosis for the disease. The prediction may characterize a presence of, quantity of and/or size of the set of tumor cells or the other structural and/or functional biological entities, the diagnosis of the disease, the classification of the disease, and/or the prognosis of the disease.

A training controller 140 can execute code to train the machine-learning model and/or the other machine-learning model(s) using one or more training datasets 145. Each training dataset 145 can include a set of training biomedical images from images 130a-n. Each of the biomedical images may include a digital pathology image, CT image, MRI image, ultrasound image, etc. that depicts one or more biological objects (e.g., a set of cells of one or more types). Each of the biomedical images may depict a portion of a sample, such as a tissue sample (e.g., colorectal, bladder, breast, pancreas, lung, or gastric tissue), a blood sample or a urine sample. In some instances, each of one or more of the biomedical images depicts a plurality of tumor cells or a plurality of other structural and/or functional biological entities. The training dataset 145 may have been collected (for example) from the image generation system 105.

The training controller 140 can identify an indication of a representative distribution of characteristics of a disease. The analysis system 135 may access and analyze one or more databases of clinical data associated with the disease to determine the representative distribution of the characteristics and communicate the indication of the representative distribution to the training controller 140. Alternatively, the training controller 140 may receive the indication via a user input from a remote system 150, which may be associated with (for example) a physician, nurse, hospital, pharmacist, etc. associated. The representative distribution can be correspond to realistic percentages of the various characteristics with respect to each other. For instance, a particular variant of a disease may be present in a certain percentage of subjects who have the disease. So, the representative distribution can indicate the certain percentage for the particular variant. Other characteristics can include a relapse rate of the disease, international prognostic index risk factors, and/or demographic factors of the disease. In addition to clinical characteristics, the representative distribution can also include technical characteristics, which can specify equipment and process-related characteristics. For example, technical characteristics may involve types of staining protocols (e.g., types of stains, and/or numbers of stains) used by the staining system 120, types of scanners of the imaging system 125, one or more types of data acquisition methods of the imaging system 125, etc. The technical characteristics may be determined based on the disease, since different diseases may be visualized better using a particular scanner or staining protocol.

The training controller 140 can generate the training dataset 145 to have a distribution of characteristics that corresponds to the representative distribution of the characteristics of the disease. The distribution may correspond to the representative distribution in a manner such that one or more measured properties of the distribution of the training dataset 145 are sufficiently close to (e.g., within a predefined absolute value from, within a predefined percentage from, etc.) the representative distribution, an integral (or normalized integral) of an overlap of the distribution and the representative distribution exceeds a threshold, etc. Generating the training dataset 145 can involve the training controller 140 defining or selecting a set of the images 130 that have the representative distribution. For example, breast cancer carcinoma may have a representative distribution of a ductal subtype occurring in 80-85% of cases and a lobular and other rare subtypes occurring in 15-20% of cases. So, the images included in the training dataset 145 can have a distribution of characteristics equal to or sufficiently close to (e.g., within 1%) the distribution of characteristics indicated in the representative distribution.

In some instances, the training controller 140 may determine that the images 130 lack representative data for one or more characteristics of the disease. For instance, if the disease is breast cancer, and the representative distribution can indicate that 1% of breast cancer cases involve a biopsy that stains positive for a lobular subtype. But, the training controller 140 may determine that the images 130 lack a combination of characteristics of a biopsy that stains positive for the lobular subtype that can make up 1% of the training dataset 145. Upon making this determination, the training controller 140 may output a notification to the remote system 150 indicating that the images 130 do not include representative data for the particular combination of characteristics. The training controller 140 can then receive, from the remote system 150, an adjustment for a value of the characteristic(s). For example, the adjustment may indicate that 0.5%, rather than 1%, of the training dataset 145 is to include a biopsy that stains positive for the lobular subtype. Based on the adjustment, the training controller 140 can generate the training dataset 145.

The computing system 100 can include a label mapper 155 that maps the images 130 from the imaging system 125 containing tumor cells or other structural and/or functional biological entities associated with the disease to a “tumor” label and that maps images 130 not containing tumor cells or other structural and/or functional biological entities associated with the disease to a “non-tumor” label. Mapping data may be stored in a mapping data store (not shown). The mapping data may identify each image that is mapped to either of the tumor label or non-tumor label.

In some instances, labels associated with the training dataset 145 may have been received or may be derived from data received from the remote system 150. The received data may include (for example) one or more medical records corresponding to a particular subject to which one or more of the images 130 corresponds. The medical records may indicate (for example) a professional's diagnosis or characterization that indicates, with respect to a time period corresponding to a time at which one or more input image elements associated with the subject were collected or a subsequent defined time period, whether the subject had a tumor and/or a stage of progression of the subject's tumor (e.g., along a standard scale and/or by identifying a metric, such total metabolic tumor volume (TMTV)). The received data may further include the pixels of the locations of tumors or tumor cells within the one or more images associated with the subject. Thus, the medical records may include or may be used to identify, with respect to each training image, one or more labels. In some instances, images or scans that are input to one or more classifier subsystems are received from the remote system 150. For example, the remote system 150 may receive images 130 from the image generation system 105 and may then transmit the images 130 or scans (e.g., along with a subject identifier and one or more labels) to the analysis system 135.

Training controller 140 can use the mappings of the training dataset 145 to train a machine-learning model. More specifically, training controller 140 can access an architecture of a model, define (fixed) hyperparameters for the model (which are parameters that influence the learning process, such as e.g. the learning rate, size/complexity of the model, etc.), and train the model such that a set of parameters are learned. More specifically, the set of parameters may be learned by identifying parameter values that are associated with a low or lowest loss, cost, or error generated by comparing predicted outputs (obtained using given parameter values) with actual outputs. In some instances, a machine-learning model can be configured to iteratively fit new models to improve estimation accuracy of an output (e.g., that includes a metric or identifier corresponding to an estimate or likelihood as to portions of the image that include depictions of tumor cells or other structural and/or functional biological entities). Using a training dataset with the distribution corresponding to the representative distribution to train the machine-learning model may result in a trained machine learning model that can more accurately detect depictions of tumor cells or other structural and/or functional biological entities associated with the disease than a machine-learning model trained with a training dataset that is different than the representative distribution.

A machine learning (ML) execution handler 160 can use the architecture and learned parameters to process non-training data and generate a result. For example, ML execution handler 160 may access biomedical image not represented in the training dataset 145. For example, the biomedical image may be a histopathological image of a slice of specimen not represented in the training dataset 145. In some embodiments, the biomedical image generated is stored in a memory device. The image may be generated using the imaging system 125. In some embodiments, the image is generated or obtained from a microscope or other instrument capable of capturing image data of a specimen-bearing microscope slide, as described herein. In some embodiments, the biomedical image is generated or obtained using a 2D scanner, such as one capable of scanning image tiles. Alternatively, the image may have been previously generated (e.g. scanned) and stored in a memory device (or, for that matter, retrieved from a server via a communication network).

In some instances, the biomedical image may be fed into a trained machine learning model having an architecture (e.g., U-Net) used during training and configured with learned parameters. The trained machine learning model may or may not have been trained with the training dataset 145 having a distribution of characteristics corresponding to the representative distribution. The trained machine learning model can output a prediction of whether or not the image depicts tumor cells or other structural and/or functional biological entities associated with the disease.

A validation controller 165 can feed one or more biomedical images into the trained machine learning model to evaluate performance metrics for the predictions output by the trained machine learning model. For instance, one or more validation datasets 170 of biomedical images may be generated to test an accuracy, precision, sensitivity, and/or F-score of the trained machine learning model in predicting the depictions of tumor cells for the disease. Each validation dataset 170 can include a set of validation biomedical images from images 130a-n. In some instances, each of one or more of the biomedical images depicts a plurality of tumor cells or a plurality of other structural and/or functional biological entities. The validation dataset 170 may have been collected (for example) from the image generation system 105.

The validation controller 165 can generate the validation dataset 170 to have a distribution of characteristics corresponding to the representative distribution of the characteristics of the disease. The distribution can correspond to the representative distribution such that one or more measured properties of the distribution of the validation dataset 170 are sufficiently close to the representative distribution. Since the representative distribution may change over time, the validation dataset 170 can be evaluated and modified, as necessary. In some instances, the validation controller 165 may perform a gap analysis between the representative distribution and the validation dataset 170 to evaluate the validation dataset 170. For example, upon generating the validation dataset 170, the validation controller 165 may determine the distribution of the characteristics of the disease in the validation dataset 170. The validation controller 165 can then compare the representative distribution to the determined distribution for the validation dataset 170 and determine whether there are any differences between the representative distribution and the determined distribution. If there is a difference, the validation controller 165 can modify the validation dataset 170 to mitigate the difference. For example, if the representative distribution involves 50% of diffuse large B-cell lymphoma cases being a germinal center B-cell subtype and 50% being an activated B-cell subtype and the validation controller 165 determines that 45% of the images in the validation dataset 170 are for the germinal center B-cell subtype and 55% are for the activated B-cell subtype, the validation controller 165 can modify the validation dataset 170 to include 50% images for the germinal center B-cell subtype and 50% images for the activated B-cell subtype.

Similar to the training dataset 145, if the validation controller 165 determines that the images 130a-n do not include representative data for a characteristic, the validation controller 165 may output a notification to the remote system 150 and receive an adjustment for an amount of the characteristic in the validation dataset 170. The validation controller 165 can then generate the validation dataset 170 accordingly.

In some instances, the ML execution handler 160 can access the validation dataset 170 and process the validation dataset 170 using the trained machine learning model. For each image in the validation dataset 170, the ML execution handler 160 can generate a prediction of a depiction of tumor cells or other structural and/or functional biological entities of the disease in the image. The validation controller 165 can compare the predictions to ground truths of the images depicting tumor cells or other structural and/or functional biological entities (e.g., based on the labels generated by the label mapper 155). Based on the comparison, the validation controller 165 can determine performance metrics for the trained-machine learning model. The performance metrics can include an accuracy, precision, sensitivity, and/or F-score of the trained machine learning model in predicting the depictions of tumor cells or other structural and/or functional biological entities for the disease.

The validation controller 165 may identify a threshold criterion for a metric associated with the prediction. For example, the threshold criterion may be a lower limit of 0.8 for the accuracy of predicting a depiction of tumor cells or other structural and/or functional biological entities. If the validation controller 165 determines that the trained machine learning model satisfies the threshold criterion by exceeding 0.8 for the accuracy, the validation controller 165 can avail the trained machine learning model for subsequent processing of biomedical images. For instance, the validation controller 165 may make the trained machine learning model available to other entities or systems for processing biomedical images associated with the disease. Once availed, the trained machine learning model can receive biomedical images and output predictions of the biomedical images depicting tumors cells or other structural and/or functional biological entities.

Alternatively, if the validation controller 165 determines that the metric does not satisfy the threshold criterion, the validation controller 165 may still avail the trained machine learning model for subsequent processing of biomedical images, but the subsequent processing of biomedical images may result in the prediction and a confidence level of the prediction being output by the trained machine learning model. The confidence level may be quantitative (e.g., a percentage or decimal) or qualitative (e.g., an indication of low, medium, or high). Outputting the confidence level can allow a user to decide whether the prediction is to be trusted or whether additional processing is to be performed for a biomedical image before a determination of the presence of tumor cells or other structural and/or functional biological entities can be made.

In some instances, once the trained machine learning model is availed for subsequent processing of biomedical images and the subsequent processing of a biomedical image has occurred by the ML execution handler 160, an image characterizer 175 identifies a predicted characterization for the biomedical image based on the execution of the image processing. The execution may itself produce a result that includes the characterization, or the execution may include results that image characterizer 175 can use to determine a predicted characterization of the specimen. For example, the subsequent processing may include characterizing a presence, quantity of, and/or size of a set of tumor cells or other structural and/or functional biological entities predicted to be present in the biomedical image. The subsequent processing may additionally or alternatively include characterizing other structural and/or functional biological entities predicted to be present in the biomedical image, the diagnosis of the disease predicted to be present in the biomedical image, the classification of the disease predicted to be present in the biomedical image, and/or the prognosis of the disease predicted to be present in the biomedical image. Image characterizer 175 may apply rules and/or transformations to map the probability and/or confidence to a characterization. As an illustration, a first characterization may be assigned if a result includes a probability greater than 50% that the biomedical image includes a set of tumor cells, and a second characterization may be otherwise assigned.

A communication interface 180 can collect results and communicate the result(s) (or a processed version thereof) to a user device (e.g., associated with a laboratory technician or care provider) or other system. For example, the results may be communicated to the remote system 150. In some instances, the communication interface 180 may generate an output that identifies the presence of, quantity of and/or size of the set of tumor cells or other structural and/or functional biological entities, the diagnosis of the disease, the classification of the disease, and/or the prognosis of the disease. The output may then be presented and/or transmitted, which may facilitate a display of the output data, for example on a display of a computing device. The result may be used to determine a diagnosis, a treatment plan, or to assess an ongoing treatment for the tumor cells.

### III. Example Use Cases

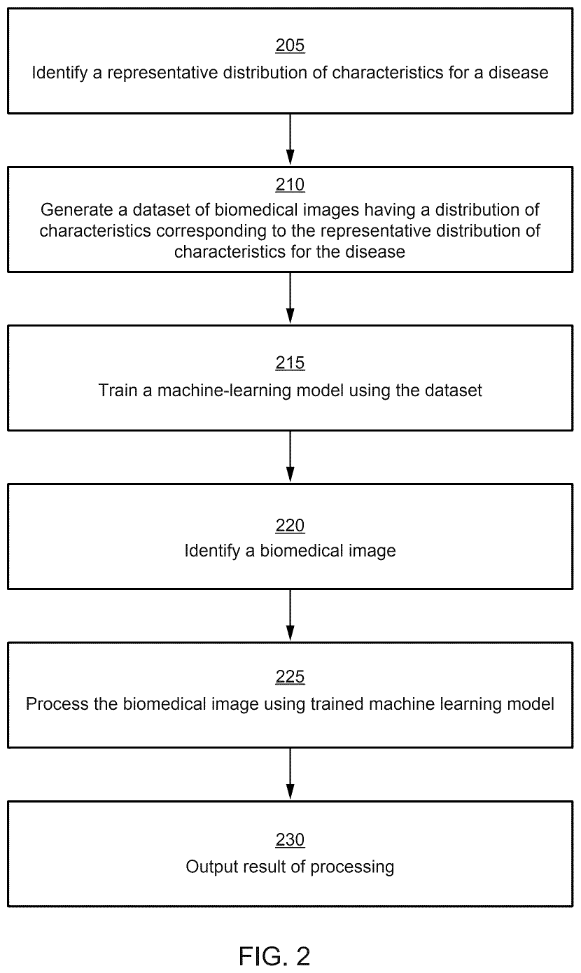

FIG. 2 illustrates an exemplary process of training and using a machine-learning model with a representative dataset. Steps of the process may be performed by one or more systems. Other examples can include more steps, fewer steps, different steps, or a different order of steps.

At block 205, a representative distribution of characteristics for a disease is identified. The representative distribution can be determined based on clinical data associated with the disease. The characteristics can involve clinical characteristics of the disease and technical characteristics associated with image generation of slices of specimens. For example, the clinical characteristics can be variants or subtypes of the disease and international prognostic index risk factors. As a particular example, the disease may be diffuse large B-cell lymphoma and clinical characteristics can involve an activated B-cell subtype, a germinal center B-cell subtype, an anaplastic variant, an immunoblastic variant, a centroblastic variant, a relapse rate, an age of a subject over sixty years, an elevated serum lactate dehydrogenase level, and/or a stage three of four of the disease.

At block 210, a dataset of biomedical images having a distribution of characteristics corresponding to the representative distribution of characteristics is generated. If the dataset includes histopathological images, each histopathological image can be stained with one or more stains for determining a characterization of a set of tumor cells. The dataset can be selected from one or more databases of biomedical images. If representative data for a particular characteristic is not available in the databases, an adjustment to a value for the characteristic can be made so that the dataset remains representative for the other characteristics.

At block 215, a machine-learning model is trained using the dataset. The machine-learning model can be a deep neural network and/or a convolutional neural network. The machine-learning model can be trained to output a prediction that a biomedical image includes a depiction of a set of tumor cells or other structural and/or functional biological entities, is associated with a diagnosis of the disease, is associated with a classification of the disease, and/or is associated with a prognosis for the disease. The training can result in a trained machine learning model.

At block 220, a biomedical image is identified. The biomedical image can be an image not included in the training dataset. The biomedical image can be identified in a request to process the biomedical image using the trained machine learning model to determine whether the biomedical image is predicted to include a depiction of tumor cells or other structural and/or functional biological entities. In some instances, the biomedical image may be part of a validation or testing dataset that can be processed by the trained machine learning model to determine performance metrics of the trained machine learning model.

At block 225, the biomedical image is processed using the trained machine learning model. The trained machine learning model may be availed for subsequent processing after the training and the subsequent processing can involve processing the biomedical image. For instance, availing the trained machine learning model may involve providing the trained machine learning model to other entities or systems for processing biomedical images associated with the disease. If the biomedical image is part of a validation or testing dataset, other biomedical images in the validation or testing dataset can also be processed using the trained machine learning model. Prior to processing the validation or testing dataset, a gap analysis can be performed to compare a distribution of characteristics for the dataset to the representative distribution. Modifications can be made to the dataset until the dataset has the representative distribution or is within an acceptable range of the representative distribution for each characteristic.

At block 230, a result of the processing is output. For example, the result may be transmitted to another device (e.g., associated with a care provider) and/or displayed. The result can correspond to a predicted characterization of the specimen of each of the biomedical images. The result can characterize a presence of, quantity of, and/or size of the set of tumor cells or the other structural and/or functional biological entities, the diagnosis of the disease, the classification of the disease, and/or the prognosis of the disease in each of the biomedical images. If the biomedical image is part of a validation or testing dataset, a performance metric can be evaluated based on the result. If the performance metric is determined to not satisfy a threshold criterion for the validation or testing dataset, the result may also include a confidence level of the prediction.

### IV. Exemplary Characteristics and Results

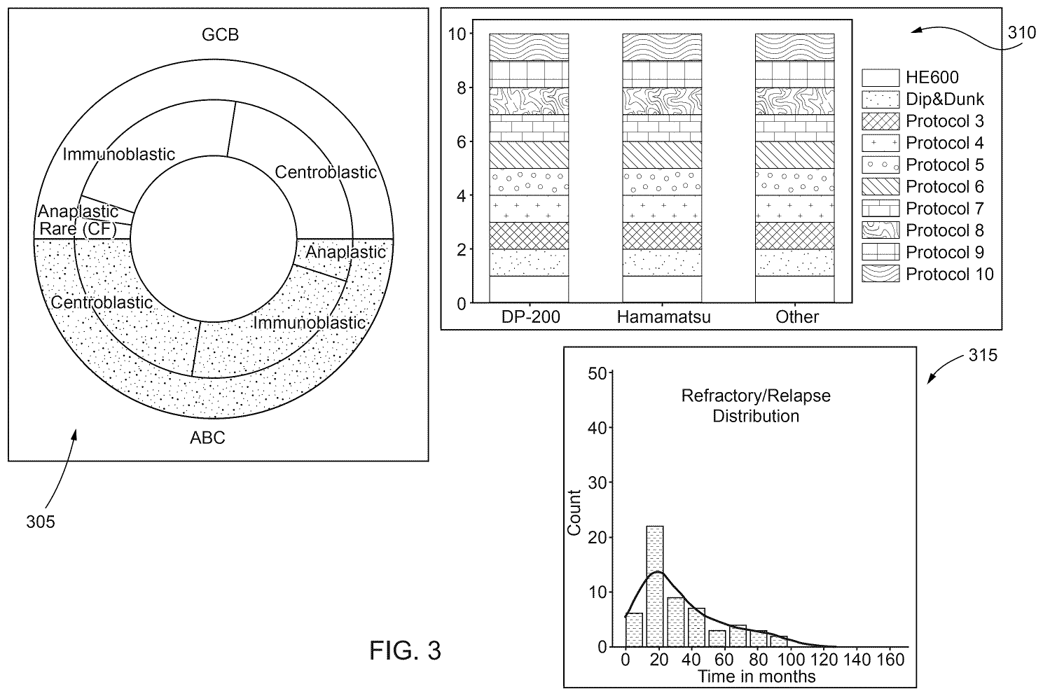

FIG. 3 shows exemplary characteristics of a representative dataset for training or testing a machine-learning model. The characteristics are for diffuse large B-cell lymphoma and include a first graph 305 of a representative distribution of subtypes and variants of diffuse large B-cell lymphoma, a second graph 310 of scanners and staining protocols for image generation, and a third graph 315 of a distribution of refractory/relapse of diffuse large B-cell lymphoma. Additional characteristics may additionally be considered.

The first graph 305 shows the representative distribution of diffuse large B-cell lymphoma as including a germinal center B-cell subtype and an activated B-cell subtype each making up 50% of diffuse large B-cell lymphoma cases. In each subtype, a centroblastic variant and an immunoblastic variant make up approximately 22% of the cases. For the germinal center B-cell subtype, an anaplastic variant makes up approximately 3% of the cases and a rare variant makes up the remaining approximately 3% of cases. For the activated B-cell subtype, the anaplastic variant makes up approximately 6% of the cases. So, a training, testing, or validation dataset for a machine-learning model can involve histopathological images having a distribution that corresponds to these respective percentages.

The second graph 310 shows three different scanners, a DP-200 scanner, a Hamamatsu scanner, and another scanner. Each of the scanners can scan slides of slices of specimen to generate histopathological images. The slides can be stained with ten different staining protocols. So, a representative distribution may specify one or more of the thirty different combinations of scanners and staining protocols that are to be used for the histopathological images of the training, testing, or validation dataset. For example, the representative distribution may indicate that 30% of the histopathological images of the dataset are to involve a HE600 staining protocol and be generated using the DP-200 scanner and 70% of the histopathological images of the dataset are to involve staining Protocol 8 and be generate using the Hamamatsu scanner.

The third graph 315 shows the representative distribution of a refractory/relapse rate for diffuse large B-cell lymphoma over an eight year period. Each bin of the third graph 315 represents a one-year period. The representative distribution indicates relapse is most likely to occur in the second year and least likely to occur in the fourth and eighth year. So, the training, testing, or validation dataset for the machine-learning model can involve histopathological images of subjects having a similar distribution of refractory/relapse.

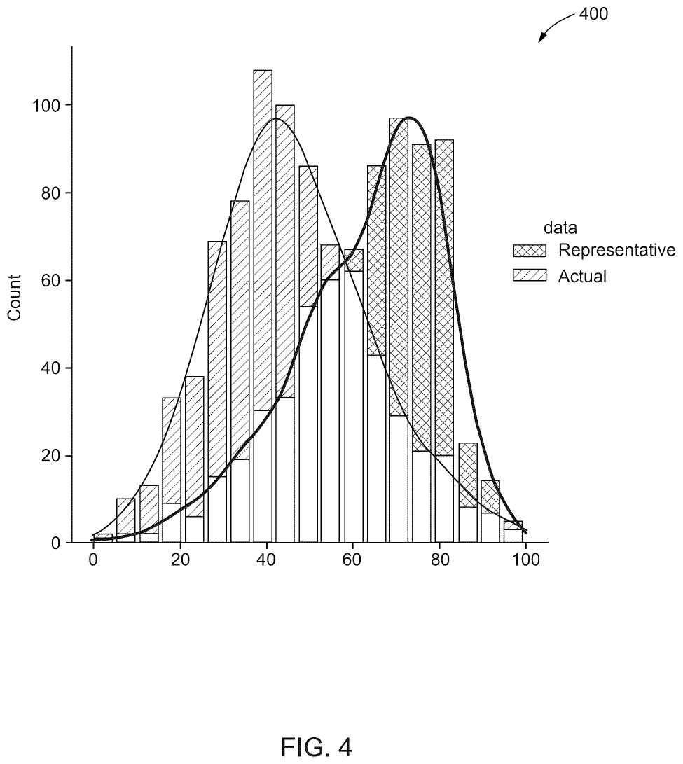

FIG. 4 shows a graph 400 of exemplary results of a gap analysis between a representative distribution and an actual distribution in a generated dataset for a characteristic of a disease. For instance, the characteristic may be an age at diagnosis of the disease. The representative distribution indicates that a majority of cases of the disease are diagnosed between the ages of thirty-five and forty-five. But, the actual distribution shows that a generated dataset of biomedical images of the disease indicates that a majority of cases of the disease are diagnosed between the ages of sixty-five to eighty-five. Upon performing the gap analysis, the generated dataset may be modified so that the actual distribution more closely matches the representative distribution for the age characteristic.

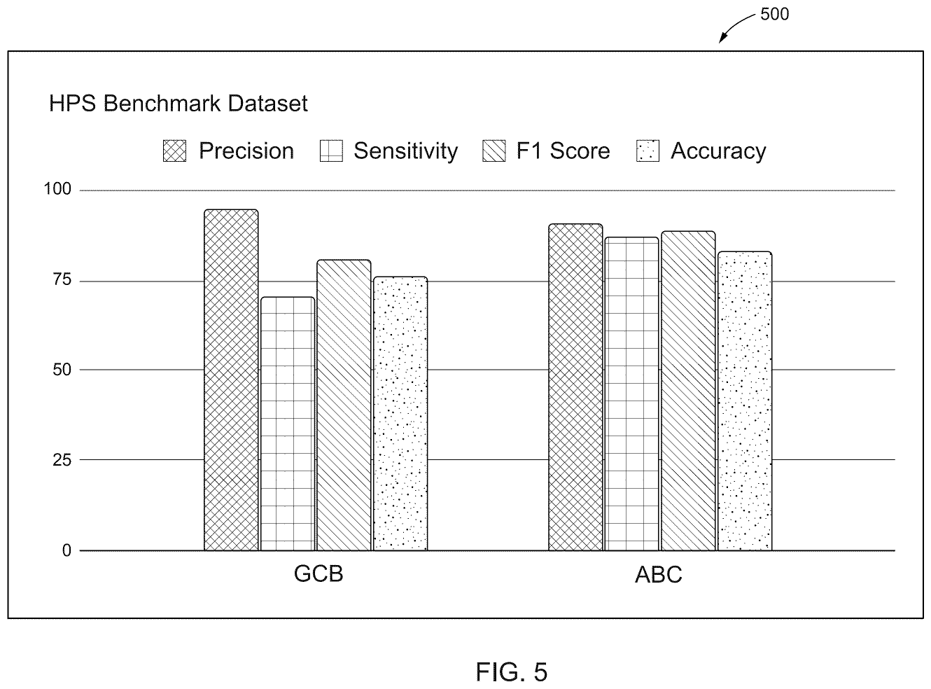

FIG. 5 shows a graph 500 of exemplary performance metrics for a machine-learning model tested using a representative dataset. The performance metrics involve precision, sensitivity, an F1 score, and accuracy of predicting a depiction of germinal center B-cell tumor cells and activated B-cell tumor cells in histopathological images of diffuse large B-cell lymphoma. The representative dataset includes histopathological images having a distribution of characteristics that corresponds to a representative distribution for characteristics of germinal center B-cell and activated B-cell. As illustrated in the graph 500, the machine-learning model has an accuracy, precision, and F1 score for predicting depictions of tumor cells for germinal center B-cell diffuse large B-cell lymphoma and activated B-cell diffuse large B-cell lymphoma above 75%. In addition, the machine-learning model has a sensitivity above 75% for activated B-cell diffuse large B-cell lymphoma. But, the sensitivity of the machine-learning model at predicting the presence of tumor cells is less than 75% for germinal center B-cell diffuse large B-cell lymphoma. There may be a threshold criterion for the machine-learning model of exceeding 75% for the sensitivity. So, upon determining that the sensitivity is less than 75% for germinal center B-cell diffuse large B-cell lymphoma, subsequent processing of histopathological images by the machine-learning model may result in the prediction of the presence of tumor cells being output along with a confidence level of the prediction.

### V. Additional Considerations

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification, and variation of the concepts herein disclosed may be resorted to by those skilled in the art, and that such modifications and variations are considered to be within the scope of this invention as defined by the appended claims.

The description provides preferred exemplary embodiments only, and is not intended to limit the scope, applicability or configuration of the disclosure. Rather, the description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

1. A computer-implemented method comprising:
identifying a representative distribution of a plurality of characteristics for a disease;
generating a dataset comprising a set of biomedical images, wherein a distribution of the plurality of characteristics for the dataset corresponds to the representative distribution of the plurality of characteristics for the disease;
processing the dataset using a trained machine learning model; and
outputting a result of the processing, wherein the result corresponds to a prediction that a biomedical image of the dataset includes a depiction of a set of tumor cells or other structural and/or functional biological entities associated with the disease, the biomedical image is associated with a diagnosis of the disease, the biomedical image is associated with a classification of the disease, and/or the biomedical image is associated with a prognosis for the disease.

2. The computer-implemented method of claim 1, further comprising, prior to processing the dataset using the trained machine learning model:
determining the distribution of the plurality of characteristics in the dataset;
determining a difference between the distribution and the representative distribution; and
modifying the dataset based on the difference.

3. The computer-implemented method of claim 1, further comprising:
identifying a threshold criterion for a metric associated with the prediction;
determining that the metric satisfies the threshold criterion; and
availing the trained machine learning model for subsequent processing of biomedical images.

4. The computer-implemented method of claim 3, further comprising:
determining that the metric does not satisfy the threshold criterion;
availing the trained machine learning model for subsequent processing of biomedical images;
performing the subsequent processing of another set of biomedical images using the trained machine learning model; and
outputting another result of the subsequent processing, wherein the other result indicates the prediction and a confidence level of the prediction based on the metric not satisfying the threshold criterion.

5. The computer-implemented method of claim 1, wherein the prediction characterizes a presence of, quantity of and/or size of the set of tumor cells or the other structural and/or functional biological entities, the diagnosis of the disease, the classification of the disease, and/or the prognosis of the disease.

6. The computer-implemented method of claim 1, wherein the plurality of characteristics comprise clinical characteristics and technical characteristics, wherein the clinical characteristics include one or more variants of the disease, a relapse rate, one or more international prognostic index risk factors, and/or one or more demographic factors, and wherein the technical characteristics include one or more types of data acquisition methods, one or more types of scanner, and/or one or more types of staining protocol.

7. A computer-implemented method comprising:
identifying a representative distribution of a plurality of characteristics for a disease;
generating a dataset comprising a set of biomedical images, wherein a distribution of the plurality of characteristics for the dataset corresponds to the representative distribution of the plurality of characteristics for the disease;
training a machine-learning model using the dataset, wherein an outcome of the training corresponds to a trained machine learning model; and
availing the trained machine learning model to process a biomedical image.

8. The computer-implemented method of claim 7, further comprising, prior to training the machine-learning model:
determining a plurality of biomedical images including the set of biomedical images excludes representative data for a characteristic of the plurality of characteristics;
outputting a notification indicating the plurality of biomedical images excludes the representative data;
receiving an adjustment for a value of the characteristic for the representative distribution; and
generating the dataset comprising the adjustment for the value of the characteristic.

9. The computer-implemented method of claim 7, wherein the dataset is a first dataset and further comprising:
processing a second dataset of biomedical images using the trained machine learning model; and
outputting a result of the processing, wherein the result corresponds to a prediction that a biomedical image of the second dataset includes a depiction of a set of tumor cells or other structural and/or functional biological entities associated with the disease, the biomedical image is associated with a diagnosis of the disease, the biomedical image is associated with a classification of the disease, and/or the biomedical image is associated with a prognosis for the disease.

10. The computer-implemented method of claim 9, wherein the distribution of the plurality of characteristics is a first distribution and further comprising, prior to processing the second dataset using the trained machine-learning:
determining a second distribution of the plurality of characteristics in the second dataset;
determining a difference between the second distribution and the representative distribution; and
modifying the second dataset based on the difference.

11. The computer-implemented method of claim 9, further comprising:
identifying a threshold criterion for a metric associated with the prediction;
determining, based on the processing of the second dataset, that the metric satisfies the threshold criterion; and
availing the trained machine learning model for subsequent processing of biomedical images.

12. The computer-implemented method of claim 11, further comprising:
determining, based on the processing of the second dataset, that the metric does not satisfy the threshold criterion;
availing the trained machine learning model for subsequent processing of biomedical images;
performing the subsequent processing biomedical images using the trained machine learning model; and
outputting another result of the subsequent processing, wherein the other result indicates the prediction and a confidence level of the prediction based on the metric not satisfying the threshold criterion.

13. The computer-implemented method of claim 9, wherein the prediction characterizes a presence of, quantity of and/or size of the set of tumor cells or the other structural and/or functional biological entities, the diagnosis of the disease, the classification of the disease, and/or the prognosis of the disease.

14. The computer-implemented method of claim 7, wherein the plurality of characteristics comprise clinical characteristics and technical characteristics, wherein the clinical characteristics include one or more variants of the disease, a relapse rate, one or more international prognostic index risk factors, and/or one or more demographic factors, and wherein the technical characteristics include one or more types of data acquisition methods, one or more types of scanner, and/or one or more types of staining protocol.

15. A computer-implemented method comprising:
identifying a biomedical image of a slice of specimen, wherein the biomedical image is associated with a disease;
inputting the biomedical image to a trained machine learning model, wherein the trained machine learning model was trained using a dataset having a distribution of a plurality of characteristics for the disease corresponding to a representative distribution of the plurality of characteristics for the disease; and
receiving a result of the trained machine learning model, wherein the result corresponds to a prediction that the biomedical image includes a depiction of a set of tumor cells or other structural and/or functional biological entities associated with the disease, the biomedical image is associated with a diagnosis of the disease, the biomedical image is associated with a classification of the disease, and/or the biomedical image is associated with a prognosis for the disease.

16. The computer-implemented method of claim 15, further comprising:
prior to inputting the biomedical image to the trained machine learning model, identifying a threshold criterion for a metric associated with the prediction;
determining that the metric satisfies the threshold criterion; and
receiving the result corresponding to the prediction that the biomedical image includes the depiction of the set of tumor cells or the other structural and/or functional biological entities associated with the disease.

17. The computer-implemented method of claim 16, further comprising:
determining that the metric does not satisfy the threshold criterion; and
outputting the result of the subsequent processing, wherein the result indicates the prediction and a confidence level of the prediction based on the metric not satisfying the threshold criterion.

18. The computer-implemented method of claim 15, wherein the prediction characterizes a presence of, quantity of and/or size of the set of tumor cells or the other structural and/or functional biological entities, the diagnosis of the disease, the classification of the disease, and/or the prognosis of the disease.

19. The computer-implemented method of claim 15, wherein the plurality of characteristics comprise clinical characteristics and technical characteristics, wherein the clinical characteristics include one or more variants of the disease, a relapse rate, one or more international prognostic index risk factors, and/or one or more demographic factors, and wherein the technical characteristics include one or more types of data acquisition methods, one or more types of scanner, and/or one or more types of staining protocol.

20. A system comprising:
one or more data processors; and
a non-transitory computer readable storage medium containing instructions which, when executed on the one or more data processors, cause the one or more data processors to perform operations comprising:
identifying a representative distribution of a plurality of characteristics for a disease;
generating a dataset comprising a set of biomedical images, wherein a distribution of the plurality of characteristics for the dataset corresponds to the representative distribution of the plurality of characteristics for the disease;
processing the dataset using a trained machine learning model; and
outputting a result of the processing, wherein the result corresponds to a prediction that a biomedical image of the dataset includes a depiction of a set of tumor cells or other structural and/or functional biological entities associated with the disease, the biomedical image is associated with a diagnosis of the disease, the biomedical image is associated with a classification of the disease, and/or the biomedical image is associated with a prognosis for the disease.

21. A computer-program product tangibly embodied in a non-transitory machine-readable storage medium, including instructions configured to cause one or more data processors to perform operations comprising:
identifying a representative distribution of a plurality of characteristics for a disease;
generating a dataset comprising a set of biomedical images, wherein a distribution of the plurality of characteristics for the dataset corresponds to the representative distribution of the plurality of characteristics for the disease;
processing the dataset using a trained machine learning model; and
outputting a result of the processing, wherein the result corresponds to a prediction that a biomedical image of the dataset includes a depiction of a set of tumor cells or other structural and/or functional biological entities associated with the disease, the biomedical image is associated with a diagnosis of the disease, the biomedical image is associated with a classification of the disease, and/or the biomedical image is associated with a prognosis for the disease.
